(19) United States
(12) Patent Application Publication
(10) Pub. No.: US 2017/0071834 A1
(43) Pub. Date: Mar. 16, 2017

(54) STABILIZED OXYRESVERATROL COMPOSITIONS

(71) Applicant: Pangaea Laboratories Limited, Hertfordshire (GB)

(72) Inventors: Kavitha Murugesan, Hertfordshire (GB); Elliot Isaacs, Hertfordshire (GB); Daniel Isaacs, Hertfordshire (GB)

(21) Appl. No.: 15/267,778

(22) Filed: Sep. 16, 2016

(30) Foreign Application Priority Data
Sep. 16, 2015 (GB) GB1516425.4

(51) Int. Cl. A61K 8/34; A61Q 19/02; A61Q 19/00; A61K 8/60; A61K 8/23

(52) U.S. Cl. CPC A61K 8/347; A61K 8/345; A61K 8/60; A61K 8/23; A61Q 19/007; A61Q 19/02; A61K 2800/882; A61K 2800/31; A61K 2800/52

(57) ABSTRACT

The invention relates to an anhydrous composition that provides stable oxyresveratrol in a cosmetically or pharmaceutically acceptable medium with butylene glycol. Preferably, the oxyresveratrol is present within the composition in a concentration in the range of between about 0.00001% and about 20%. Preferably, the butylene glycol is present in the composition in a concentration in the range of between about 0.00001% and about 30%. The anhydrous composition may consist solely of oxyresveratrol and butylene glycol.

### PRIORITY

The present application is related to, and claims the priority benefit of, Great Britain patent application serial no. GB 1516425.4, filed Sep. 16, 2015, the contents of which are incorporated into the present disclosure in their entirety.

### TECHNICAL FIELD

The present invention provides stabilised oxyresveratrol-containing compositions, and in particular stabilised oxyresveratrol-containing compositions for use in lightening skin.

### BACKGROUND

There are several routes to lightening skin using cosmetic or pharmaceutical compositions. Kojic acid is a popular ingredient of these compositions as it has been found to block several parts of the melanogenic pathway simultaneously, thereby creating significant results in a variety of skin types.

An emerging alternative option for lightening skin is oxyresveratrol. As discussed above with regards to Kojic acid, oxyresveratrol has also been found to block several parts of the melanogenic pathway. The principle problem with using oxyresveratrol is stability. Oxyresveratrol is highly reactive and when present within solution degrades within weeks. This degradation process has been found to be accelerated by heat, UV, or high pH. Degraded oxyresveratrol has little or no function for skin lightening and is a distinctive yellow colour.

Attempts to solve the stability issue have involved using very low concentrations (from 0.001 to 0.01%) of oxyresveratrol within cosmetic or pharmaceutical compositions. By using very low concentrations of oxyresveratrol it has been found that discoloration of the resulting compositions is reduced. However, the effectiveness of the cosmetic or pharmaceutical compositions for skin lightening is also reduced due to oxyresveratrol being present at such low concentrations. These prior art compositions are therefore far from ideal.

### BRIEF SUMMARY

According to a first aspect, the present invention provides an anhydrous composition comprising oxyresveratrol and butylene glycol in a cosmetically or pharmaceutically acceptable medium.

The term ‘anhydrous’ is used herein to refer to a composition or solvent(s) which is substantially free from or contains no water.

The oxyresveratrol-containing compositions of the present invention have improved stability compared to known oxyresveratrol-containing compositions. Furthermore, oxyresveratrol may be present within the compositions of the present invention at greater concentrations than known oxyresveratrol-containing compositions without having a detrimental effect on the stability of the oxyresveratrol within the compositions. The present invention therefore provides oxyresveratrol-containing compositions having improved stability and improved effectiveness for skin lightening compared to known oxyresveratrol-containing compositions.

The anhydrous composition may further comprise one or more additional anhydrous solvents such as, for example, one or more anhydrous solvent selected from: ethanol, organic solvents, oils, silicones, long-chain alcohols, ketones, ethers, and any combination thereof.

The anhydrous composition may be a cosmetically or pharmaceutically acceptable composition.

The anhydrous composition may consist solely of oxyresveratrol and butylene glycol.

According to a second aspect, the present invention provides a cosmetically or pharmaceutically acceptable formulation comprising at least a first part and a second part, in which the first part comprises an anhydrous composition comprising oxyresveratrol and butylene glycol, and in which the second part comprises one or more solvents selected from anhydrous solvents, aqueous solvents, or a combination thereof.

The first part of the formulation may comprise one or more additional anhydrous agents, for example, one or more anhydrous solvent selected from: ethanol, organic solvents, oils, silicones, long-chain alcohols, ketones, ethers, and any combination thereof. The first part of the formulation may consist solely of oxyresveratrol and butylene glycol. The second part of the composition may comprise aqueous solvents. The second part of the composition may comprise anhydrous solvents. The second part of the composition may comprise one or more solvents selected from ethanol, organic solvents, oils, silicones, long-chain alcohols, ketones, ethers, and any combination thereof.

The formulation may comprise more than two parts, for example three or more parts.

According to a further aspect, the present invention comprises a method of preparing a ready-to-use cosmetically or pharmaceutically acceptable mixed formulation for lightening skin comprising:
obtaining a cosmetically or pharmaceutically acceptable formulation consisting of at least a first part and a second part as herein described; and
mixing at least the first and second parts of the formulation together to provide the ready-to-use mixed formulation.

Oxyresveratrol may be obtained from any suitable source. For example, various plants are sources of oxyresveratrol, notably the Smilacaceae, Moraceae, Liliaceae, Cyperaceae and Myrtaceae families, particularly the species Smilax china, Artocarpus lakoocha, Ramulus mori and Morus alba. Alternatively, oxyresveratrol may be derived from any suitable methods, such as for example synthetic manufacture.

Oxyresveratrol provides a skin lightening effect at very low concentrations. Oxyresveratrol may therefore be present within the composition and/or within the cosmetically or pharmaceutically active formulation comprising at least two parts mixed together (herein referred to as the mixed formulation), at a concentration of at least 0.00001%. Preferably, oxyresveratrol is present within the anhydrous composition and/or the first part of the formulation and/or the mixed formulation, at a concentration of at least 0.01%, preferably at a concentration of at least 0.1%, more preferably at a concentrations of at least 0.5%, for example at a concentration of at least 1%. Oxyresveratrol is preferably present within the anhydrous composition and/or the first part of the formulation and/or the mixed formulation, at a concentration of no more than about 20%, preferably no more than 15%, for example no more than 10%. Oxyresveratrol is preferably present within the anhydrous composition and/or the first part of the formulation and/or the mixed formulation, at a concentration within the range of between 0.00001% and 20%, more preferably within the range of between 0.01% and 20%, especially preferably in the range of between 0.01% and 15%, for example in the range of between 0.1% and 10%. Preferably, the concentration of oxyresveratrol within the anhydrous composition and/or the first part of the formulation and/or mixed formulation is 0.5%.

The compositions and formulations of the present invention comprise butylene glycol as a solvent for oxyresveratrol. The concentration of butylene glycol present within the compositions, first and/or second parts of the formulations, and/or mixed formulations, of the present invention may vary depending on the concentration of oxyresveratrol within the compositions and/or first part of the formulation and/or second part of the formulation and/or mixed formulations. For example, in embodiments in which the compositions and/or first and/or second parts of the formulations and/or mixed formulations comprise low oxyresveratrol concentrations, similarly low butylene glycol concentrations are also present within the composition or mixed formulation. Butylene glycol may for example be present within the composition, and/or first and/or second parts of the formulations and/or mixed formulation, at a concentration of at least 0.00001%. Preferably, butylene glycol is present within the composition, and/or first and/or second parts of the formulations and/or mixed formulation at a concentration of at least 0.05%, preferably at a concentration of at least 0.5%, more preferably at a concentration of at least 5%. Butylene glycol is preferably present within the composition and/or first and/or second parts of the formulations, and/or mixed formulation at a concentration of no more than about 30%, preferably no more than about 20%. Butylene glycol is preferably present within the composition and/or first and/or second parts of the formulations, and/or mixed formulation of the present invention at a concentration in the range of between about 0.00001% and about 50%, more preferably in between about 0.05% and about 30%, for example between about 5% and about 15%. Preferably, the concentration of butylene glycol within a mixed formulation is 15%.

The ratio of the concentration of oxyresveratrol to butylene glycol within the composition, and/or first and/or second parts of the formulations, and/or mixed formulation may be any suitable ratio. For example, the concentration of oxyresveratrol to butylene glycol within the composition, and/or first and/or second parts of the formulations and/or mixed formulation may be present at a ratio of at least 1:1, preferably at a ratio of at least 1:10; up to a ratio of about 1:100, for example at a ratio of 1:30.

The compositions and/or mixed formulations of the present invention are stable over a significant period of time with minimal or significantly reduced degradation of the oxyresveratrol and/or the lightening effect of the oxyresveratrol within the compositions and/or formulations over time. The compositions and/or mixed formulations preferably exhibit minimal signs of degradation, such as for example discolouration (i.e. yellowing effect of the composition/formulation) within 24 hours, preferably within 1 week, more preferably within 1 month, even more preferably within 36 months, for example within six weeks (42 days) from preparation of the composition and/or from mixing together the at least first and second parts of the formulation.

The at least two parts of the formulation may be mixed together prior to use, such as for example immediately prior to use. Alternatively, the at least two parts of the formulation may be mixed together and stored for a significant period of time prior to use. For example, the at least two parts of the formulation may be mixed together in sufficient quantities to provide a mixed formulation which may be used for several repeated applications as part of a course of applications, and can therefore be stored for a considerable period of time between applications, such as for example up to 42 days, without significant degradation of the oxyresveratrol within the mixed formulation.

Once the at least two parts of the formulation have been mixed together to provide the mixed formulations, the mixed formulation is preferably stable over a significant period of time. Each part of the formulation of the present invention may be used, for example applied, separately in series. Use of each part in series may be separated by a suitable period of time, such as for example a number of seconds, or for example up to 6 weeks (from the initial mixing the parts and/or initial application of a part of the formulation) as part of a course of applications.

The compositions and/or mixed formulations may comprise additional agents in order to enhance the stability of the compositions and/or formulations. For example, the compositions and/or one or more parts of the formulations may comprise one or more of: preservatives, stabilisers, antioxidants, sulphur dioxide donors, or any combination thereof. Preferably, the first part of the formulation comprises one or more of: preservatives, stabilisers, antioxidants or sulphur dioxide donors, or any combination thereof. Preferably, the second part of the formulation comprises one or more of: preservatives, stabilisers, antioxidants or sulphur dioxide donors, or any combination thereof.

In one embodiment, the composition (or one or more parts of the formulation) further comprises a skin conditioning agent, such as for example gluconolactone; and/or a sulphur dioxide donor, such as for example sodium metabisulphite. Preferably, the composition (or one or more parts of the formulation) further comprises a skin conditioning agent, such as for example gluconolactone; and a sulphur dioxide donor, such as for example sodium metabisulphite. Preferably, the first part of the formulation comprises a skin conditioning agent, such as for example gluconolactone; and/or a sulphur dioxide donor, such as for example sodium metabisulphite. Preferably, the first part of the formulation comprises a skin conditioning agent, such as for example gluconolactone; and a sulphur dioxide donor, such as for example sodium metabisulphite. Preferably, the second part of the formulation comprises a skin conditioning agent, such as for example gluconolactone; and/or a sulphur dioxide donor, such as for example sodium metabisulphite. Preferably, the second part of the formulation comprises a skin conditioning agent, such as for example gluconolactone; and a sulphur dioxide donor, such as for example sodium metabisulphite.

The additional skin conditioning agent and/or sulphur dioxide donor may be present at any suitable concentration within the composition and/or formulation of the present invention. For example the additional skin conditioning agent and/or sulphur dioxide donor may each be present within the composition or mixed formulation at a concentration of between about 0.0001% and about 30%, preferably between about 0.01% and 10%, for example between about 0.1% and about 1%.

According to one embodiment, the composition or mixed formulation comprises a skin conditioning agent, for example gluconolactone, at a concentration of at least 0.05%, preferably at least 1%, for example 0.74%, at least 10%; and/or a sulphur dioxide donor, for example sodium metabisulphite, at a concentration of at least 0.01%, preferably at least 1%, more preferably at least 5%, for example at least 10%. Preferably, the composition or the formulation comprises 0.1% sulphur dioxide donor (for example sodium metabisulphite) and 1% skin conditioning agent (for example gluconolactone). The skin conditioning agent (for example gluconolactone) and/or sulphur dioxide donor (for example sodium metabisulphite) may be present within the composition and/or mixed formulation of the present invention in order to reduce degradation of the oxyresveratrol within the composition and/or formulation, however other functions of these additional agents are also considered.

In one embodiment, the mixed formulation comprises oxyresveratrol, butylene glycol, at least one skin conditioning agent (for example gluconolactone) and at least one sulphur dioxide agent (for example sodium metabisulphite), and optionally water.

In one embodiment, the formulation comprises a first part comprising an anhydrous composition comprising oxyresveratrol and butylene glycol; and a second part comprising at least one sulphur dioxide donor (for example sodium metabisulphite), at least one skin conditioning agent (for example gluconolactone), and optionally water.

In one embodiment, the mixed formulation comprises oxyresveratrol, butylene glycol, sodium metabisulphite and gluconolactone, and optionally water.

In one embodiment, the mixed formulation comprises oxyresveratrol at a concentration between about 0.00001% and 30%, (preferably between about 0.01% and 20%); butylene glycol at a concentration between about 0.00001% and 50%; (preferably between about 0.05% and 30%), at least one sulphur dioxide donor (for example sodium metabisulphite) at a concentration between about 0.00001% and 30% (preferably between 0.01% and 10%), at least one skin conditioning agent (for example gluconolactone) at a concentration between about 0.00001% and 30% (preferably between 0.1% and 10%); and the remainder water.

In one embodiment, the mixed formulation comprises oxyresveratrol at a concentration of 0.5%; butylene glycol at a concentration of 15%; at least one sulphur dioxide donor (for example sodium metabisulphite); and at least one skin conditioning agent (for example gluconolactone), and optionally water.

In one embodiment, the mixed formulation comprises oxyresveratrol at a concentration of 0.5%; butylene glycol at a concentration of 15%; water at a concentration of 83%; at least one sulphur dioxide donor (for example sodium metabisulphite) at a concentration of 0.1%; and at least one skin conditioning agent (for example gluconolactone) at a concentration of 1%.

In one embodiment, the mixed formulation comprises oxyresveratrol at a concentration of 0.5%, butylene glycol at a concentration of 15%; sodium metabisulphite at a concentration of 0.1%; gluconolactone at a concentration of 0.74%, and water.

The composition and/or formulation of the present invention may include one or more additional skin lightening components, and/or skin lightening methods, and/or skin lightening devices. Any suitable skin lightening method, and/or device, and/or composition is considered. Suitable additional skin lightening components may include, but are not limited to, one or more of: compositions capable of reflecting or absorbing UV (commonly known as sunscreens), ascorbic acid, vitamin c derivative, hydroquinone, kojic acid, lactic acid, L-leucine, niacinamide, thiotic acid, a form of arbutin, keratolytic agents such as salicylic acid, physical and chemical exfoliants, superficial, medium and/or deep chemical peels, and any combination thereof. Preferably, suitable skin lightening components include, but are not limited to, ascorbic acid, vitamin c derivative, hydroquinone, kojic acid, lactic acid, L-leucine, niacinamide, thiotic acid, a form of arbutin, and any combination thereof.

The composition and/or formulation may further comprise any other suitable pharmaceutically or cosmetically active agent (defined as a natural or synthetic compound that has a cosmetic or therapeutic effect on the skin, hair or nails) including but not limited to one or more of: lightening agents, darkening agents, anti-acne agents, shine control agents, anti-microbial agents, anti-inflammatory agents, anti-mycotic agents, anti-parasite agents, external analgesic or anaesthetic agents, sunscreens, photo-protectors, antioxidants, keratolytic agents, detergents or surfactants, moisturisers, humectants, nutrients, vitamins, energy-enhancers, growth factors, antiperspirant agents, astringents, deodorants, hair-removers, firming agents, anti-callous agents and agents for hair, nail and/or skin conditioning, or any combination thereof.

Preferably, the composition and/or formulation comprises one or more of: resveratrol, green tea, red tea, white tea, caffeine, copper peptides, copper pyrrolidone carboxylic acid (copper PCA), euk-134, copper(II) 3,5-diisopropylsalicylate, superoxide dismutase (and mimetics), dimethylmethoxy chromanol (Lipochroman-6), catalase mimetics, vitamins C, A, E, B, F, H, K (and derivatives), bacterial filtrates, or any combination of these.

The additional skin lightening or other pharmaceutically or cosmetically active agents (as described above) may be present in any suitable part of the formulation, for example the first and/or second part of the formulation prior to mixing. Preferably, the additional skin lightening and/or other pharmaceutically or cosmetically active agents are present within the first anhydrous part of the formulation prior to mixing. In some embodiments, the additional skin lightening and/or other pharmaceutically or cosmetically active agents are present within the second part of the formulation prior to mixing. The additional skin lightening and/or other pharmaceutically or cosmetically active agents may be present within the first anhydrous part and second part of the formulation prior to mixing

The compositions and/or mixed formulations and/or one or more of the at least two parts of the formulations of the present invention may be provided as an anhydrous powder, an aqueous solution, an oil-based solution, a suspension, an oil-in water mixture with or without a surfactant, a gel, or a cream. Each part of the formulation may have the same physical form, or take different physical forms.

According to a further aspect of the present invention, there is provided a kit comprising a formulation as herein described, in which the kit comprises a first supply of a first part of the formulation, and at least a second supply of a second part of the formulation.

Preferably, the kit comprises at least two separate chambers, in which a first part of the formulation as herein described is received within a first chamber, and in which a second part of the formulation as herein described is received within a second chamber.

The at least two chambers may be provided by a single container. Alternatively, the at least two chambers may be provided by at least two separate containers.

At least one of the first and second supplies of the at least two parts of the formulation may comprise one or more solid impregnated with the first and/or second parts of the formulation respectively. For example, the impregnated solid may be one or more of a cloth, tissue, or sponge.

The kit may further comprise one or more additional supplies comprising one or more additional cosmetically or pharmaceutically active compositions, which may for example lighten the skin. The one or more additional supplies may be provided within a separate chamber of the kit. The one or more additional supplies may be provided as a solid (such as for example a cloth, tissue or sponge) impregnated with the additional compositions.

The kit may further comprise one or more compositions for use with certain devices to be used in combination with the composition or formulation, with or without the device itself, and/or replacement components for the device.

For example, the kit may comprise: a composition or formulation as herein described, the kit further comprising one or more of: a sanitising solution, a sterilising solution, an analgesic or anaesthetic composition, a microneedle roller, a replacement microneedle array for the microneedle roller, a moisturising composition, and/or a sunscreen composition, and any combination thereof.

The compositions and/or mixed formulations and/or at least two parts of the compositions of the present invention may be used in combination with other procedures. In one embodiment, the compositions and/or mixed formulations may be used in conjunction with one or more devices and/or additional composition(s) to improve transdermal penetration of one or both compositions. Exemplary devices for this purpose comprise an array of microneedles such as a microneedle roller, an electrophoresis apparatus, an ultrasound emitter, an unfractionated laser, a fractionated laser, and an iontophoresis apparatus. Exemplary additional compositions considered may comprise surfactants; keratolytic agents such as salicylic acid; physical and chemical exfoliants; superficial, medium and deep chemical peels, and any combination thereof.

### BRIEF DESCRIPTION OF THE DRAWINGS

The above and other characteristics, features and advantages of the present invention will become apparent from the following detailed description, taken in conjunction with the accompanying drawing and examples, which illustrate, by way of example, the principles of the invention. This description is given for the sake of example only, without limiting the scope of the invention. The reference figures quoted below refer to the attached drawing.

FIG. 1 is a flow diagram illustrating the method of mixing together the at least two parts of the formulation to provide a ready to use mixed formulation.

### DETAILED DESCRIPTION

The present invention will be described with respect to a drawing but the invention is not limited thereto but only by the claims. The drawing described is only schematic and is non-limiting. The drawing may not include all of the features of the invention and therefore should not necessarily be considered to be an embodiment of the invention. In the drawing, the size of some of the elements may be exaggerated and not drawn to scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that operation is capable in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that operation is capable in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “an embodiment” or “an aspect” means that a particular feature, structure or characteristic described in connection with the embodiment or aspect is included in at least one embodiment or aspect of the present invention. Thus, appearances of the phrases “in one embodiment”, “in an embodiment”, or “in an aspect” in various places throughout this specification are not necessarily all referring to the same embodiment or aspect, but may refer to different embodiments or aspects. Furthermore, the particular features, structures or characteristics of any embodiment or aspect of the invention may be combined in any suitable manner, as would be apparent to one of ordinary skill in the art from this disclosure, in one or more embodiments or aspects.

Similarly, it should be appreciated that in the description various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Moreover, the description of any individual drawing or aspect should not necessarily be considered to be an embodiment of the invention. Rather, as the following claims reflect, inventive aspects lie in fewer than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some features included in other embodiments, combinations of features of different embodiments are meant to be within the scope of the invention, and form yet further embodiments, as will be understood by those skilled in the art. For example, in the following claims, any of the claimed embodiments can be used in any combination.

In the description provided herein, numerous specific details are set forth. However, it is understood that embodiments of the invention may be practised without these specific details. In other instances, well-known methods, structures and techniques have not been shown in detail in order not to obscure an understanding of this description.

In the discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The use of the term “at least one” may mean only one in certain circumstances.

The principles of the invention will now be described by a detailed description of one drawing and several examples relating to exemplary features of the invention. It is clear that other arrangements can be configured according to the knowledge of persons skilled in the art without departing from the underlying concept or technical teaching of the invention, the invention being limited only by the terms of the appended claims.

### Example 1 (Comparative Example)

### Aqueous Oxyresveratrol-Containing Compositions

In view of the teachings of the published work “Li Xu, Chao Liu, Wei Xiang, Hu Chen, Xiaoli Qin and Xianzhi Huang, 2014. Advances in the Study of Oxyresveratrol. International Journal of Pharmacology, 10: 44-54” and published UK Patent Application no. GB2497985, two aqueous oxyresveratrol-containing compositions were prepared (Compositions 1 and 2). These documents disclose oxyresveratrol-compositions comprising water and one or more organic solvents.

The first aqueous composition (Composition 1) comprises oxyresveratrol at a concentration of 0.01% (equivalent to the oxyresveratrol concentration in known prior art compositions). The second aqueous composition (Composition 2) comprises oxyresveratrol at a preferred higher concentration of 0.5%.

### Example 2

### Method for Testing the Stability of Oxyresveratrol-Containing Compositions

Each composition of Example 1 was exposed to heat and light over the course of 6 weeks to accelerate degradation of the composition in order to simulate the effects of several months under normal conditions.

As Oxyresveratrol degrades, the composition changes colour due to a yellowing effect. The colour of the composition can therefore be indicative as to the degree of degradation of the composition.

A 20 point colour scale was used to compare the colour of each of the compositions in order to determine the extent of degradation of the compositions over time. The colour scale ranged from a very pale yellow colour (labelled 1) which was indicative of a stable composition to a dark brown colour (labelled 20) indicative of a completely degraded composition.

The colour of each composition of Example 1 was compared to the colour scale after time intervals of 1 day and 42 days. The results are shown in Table 1.

TABLE 1

| Oxyresveratrol | | Colour Scale Result | |
|---|---|---|---|
| concentration | Solvent | Day 1 | Day 42 |
| 0.01% | 6% Butylene Glycol, | 1 | 3 |
| (Composition 1) | 94% Water | | |
| 0.5% | 6% Butylene Glycol, | 1 | 19 |
| (Composition 2) | 94% Water | | |

After 6 weeks, the colour of Composition 2 comprising a high concentration of oxyresveratrol (0.5%) had changed from 1 on the colour scale to 19 on the colour scale. This significant change in colour is indicative of the oxyresveratrol within the composition almost completely degrading over the 6 weeks. In contrast, after 6 weeks, the colour of Composition 1 comprising a low concentration of oxyresveratrol (0.01%) had changed from 1 on the colour scale to 3 on the colour scale. This slight change in colour is indicative of a lower volume of yellow liquid (caused by degradation of the oxyresveratrol) being present within the composition over the 6 weeks. By comparing the results, it can be seen that Composition 2 having the higher concentration of oxyresveratrol visibly degrades to a much more significant degree over the 6 week period than Composition 1 having the low concentration of oxyresveratrol (0.01%). The oxyresveratrol in Composition 1 does degrade very significantly, but the greater dilution hides this effect. It can therefore be seen from the results in Table 1 that the use of butylene glycol in water as a solvent for oxyresveratrol was not capable of inhibiting the degradation of high concentrations of oxyresveratrol within the compositions. The teachings of the prior art do not therefore provide stable oxyresveratrol-containing compositions with a desired shelf life of several months.

### Example 3 (Comparative Example)

### Anhydrous Compositions Comprising Oxyresveratrol in Propylene Glycol

It was known that the presence of water within the aqueous prior art compositions was detrimental to the stability of oxyresveratrol within the compositions. The results shown in Tables 1 and 2 also suggest that the aqueous part of the composition appears to be detrimental to the stability of the oxyresveratrol compositions over time thereby resulting in a significantly reduced shelf life of the composition.

Two anhydrous compositions comprising oxyresveratrol at two different concentrations (Composition 3: 0.01% and Composition 4: 0.5% respectively) in 100% propylene glycol were prepared.

The stability of each composition was assessed using the method described in Example 2. The results are shown in Table 2.

TABLE 2

| | | Colour Scale Result | |
|---|---|---|---|
| Oxyresveratrol | Solvent | Day 1 | Day 42 |
| 0.01% | 100% Propylene Glycol | 1 | 3 |
| (Composition 3) | | | |
| 0.5% | 100% Propylene Glycol | 4 | 18 |
| (Composition 4) | | | |

Oxyresveratrol is readily soluble in propylene glycol. Propylene glycol is a good solvent for stabilising ascorbic acid (another unstable skin lightening ingredient) as it helps to stabilise ascorbic acid. It was therefore considered that the use of propylene glycol as a solvent would help to stabilise oxyresveratrol-containing compositions.

As shown in Table 2, after 6 weeks the colour of Composition 4 comprising a high concentration of oxyresveratrol (0.5%) had changed from 4 on the colour scale to 18 on the colour scale. This significant change in colour is indicative of the oxyresveratrol within the composition almost completely degrading over the 6 weeks. In contrast, after 6 weeks, the colour of composition 3 comprising a low concentration of oxyresveratrol (0.01%) had changed from 1 on the colour scale to 3 on the colour scale. This slight change in colour is indicative of a lower volume of yellow liquid being present within the composition (as a result of oxyresvertrol degradation) over the 6 weeks cover to the results for Composition 4. By comparing the results, it can be seen that Composition 4 having the higher concentration of oxyresveratrol visibly degrades to a much more significant degree over the 6 week period than Composition 3 having the low concentration of oxyresveratrol (0.01%).

The presence of propylene glycol as a solvent did not provide any significant improvement to the stability of the oxyresveratrol-containing compositions when compared to the results shown in Table 1. The use of propylene glycol as an anhydrous solvent was not sufficient to provide a stable oxyresveratrol-containing composition having the desired shelf life of several months.

### Example 4

### Anhydrous Oxyresveratrol Compositions

After the unexpected failure of an anhydrous composition using propylene glycol, a number of other anhydrous oxyresveratrol compositions were prepared. It was expected that these anhydrous compositions would show significant improvements in oxyresveratrol stability compared to the known prior art compositions as a result of the absence of water.

The anhydrous compositions were prepared comprising a solvent selected from butylene glycol, isopentyldiol and hexylene glycol (as shown in Table 3). The stability of each composition was assessed using the method described in Example 2. The results are shown in Table 3.

TABLE 3

| Oxyresveratrol | Solvent | Day 1 | Day 42 |
|---|---|---|---|
| 0.01% | 100% Butylene Glycol | 1 | 2 |
| (Composition 5) | | | |
| 0.5% | 100% Butylene Glycol | 4 | 9 |
| (Composition 6) | | | |
| 0.01% | 100% Isopentyldiol | 1 | 3 |
| (Composition 7) | | | |
| 0.5% | 100% Isopentyldiol | 4 | 15 |
| (Composition 8) | | | |
| 0.01% | 100% Hexylene Glycol | 1 | 3 |
| (Composition 9) | | | |
| 0.5% | 100% Hexylene Glycol | 4 | 18 |
| (Composition 10) | | | |

After 6 weeks, the colour of the composition comprising a high concentration of oxyresveratrol (0.5%) in isopentyldiol (Composition 8) and hexylene glycol (Composition 10) had changed from 4 on the colour scale to 15 and 18 respectively on the colour scale. This significant change in colour is indicative of the oxyresveratrol within the compositions almost completely degrading over the 6 weeks. These results were similar to the results obtained for aqueous oxyresveratrol containing compositions (see Table 1). It can therefore be seen from Compositions 8 and 10 that the absence of water alone is not sufficient in itself to provide a stable oxyresveratrol containing composition.

In contrast, after 6 weeks, the colour of composition 6 comprising a high concentration of oxyresveratrol (0.5%) in anhydrous butylene glycol had changed from 4 on the colour scale to 9 on the colour scale. This significantly smaller change in colour of the composition is indicative of a much lower volume of yellow liquid being present within the composition 6, as a result of degradation of the oxyresveratrol, over the 6 weeks in comparison to compositions comprising isopentyldiol and hexylene glycol as solvents.

Furthermore, by comparing the results with the results in Table 1, it can be seen that the stability of Composition 6 comprising 0.5% oxyresveratrol in anhydrous butylene glycol (colour scale 9) over a 6 week period is significantly improved compared to Composition 2 comprising 0.5% oxyresveratrol in water and butylene glycol (colour scale 19). It can therefore be seen that the stability of a high concentration oxyresveratrol composition is significantly improved by providing an anhydrous composition comprising butylene glycol. This result was entirely unexpected when comparing the result of Composition 6 to the results obtained for the other anhydrous compositions (Compositions 8 and 10) comprising other anhydrous solvents of a very similar chemical structure.

The present invention provides oxyresveratrol-containing compositions with improved stability over time compared to known oxyresveratrol-containing compositions. The present invention also provides compositions comprising a higher concentration of oxyresveratrol with improved stability over time compared to known oxyresveratrol-containing compositions. The present invention also provides oxyresveratrol-containing compositions with improved skin lightening abilities having improved shelf life of over several months compared to known oxyresveratrol-containing compositions.

### Example 5

### Two Part Formulations Comprising Anhydrous Oxyresveratrol Containing Compositions

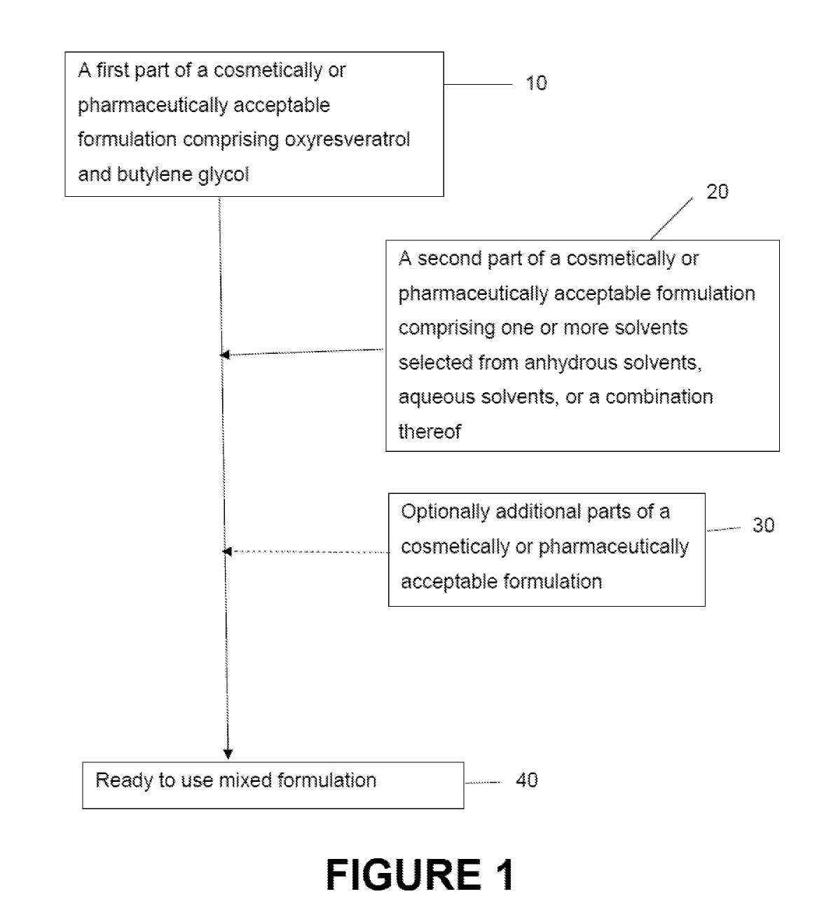

As shown in Table 3, improved stability of oxyresveratrol can be obtained by providing an anhydrous composition comprising butylene glycol. A number of two part formulations comprising anhydrous oxyresveratrol containing compositions were prepared as shown in Table 4. The anhydrous oxyresveratrol-containing composition formed the first part of the formulation. The first part 10 of the formulation was kept separate from a second part 20 of the formulation until mixed as required by the end user. The second part could be anhydrous, or aqueous. The first part of the formulation 10 and the second part of the formulation 20 (and any additional parts of the formulation 30) are mixed prior to use to provide a ready to use formulation 40 as shown in FIG. 1.

Composition 11 comprises a formulation comprising a first part comprising a composition comprising oxyresveratrol and butylene glycol, and a second part comprising water. Once the first and second parts are mixed together, the resultant mixed formulation comprises 0.5% oxyresveratrol, 15% butylene glycol and 84.5% water.

Composition 12 comprises a formulation comprising a first part comprising a composition comprising oxyresveratrol and butylene glycol, and a second part comprising water, sodium metabisulphite, and gluconolactone. Once the first and second parts are mixed together, the resultant mixed formulation comprises 0.5% oxyresveratrol, 15% butylene glycol, 0.1% sodium metabisulphite, 1.0% gluconolactone, and 83% water.

The stability of the formulation after mixing was investigated using the method described in Example 2.

A user will preferably use the mixed formulation repeatedly at separate intervals over the course of a period of 1.5 months from the initial mixing of the first and second parts of the formulation. The stability of the mixed formulations was therefore tested after a period of one day and a period of 42 days, from the initial mixing. The results are shown in Table 4.

TABLE 4

| Oxyresveratrol | Mixed Composition | Day 1 | Day 42 |
|---|---|---|---|
| 0.5% | 15% Butylene Glycol, | 1 | 15 |
| (Composition 11) | 84.5% Water, | | |
| 0.5% | 15% Butylene Glycol, | 1 | 13 |
| (Composition 12a) | 84.5% Water, | | |
| | 0.1% Sodium Metabisulphite | | |
| 0.5% | 15% Butylene Glycol, | 1 | 6 |
| (Composition 12) | 83% Water, | | |
| | 0.1% Sodium Metabisulphite, | | |
| | 1.0% Gluconolactone | | |

As shown in Table 4, it has been found that a formulation comprising a first anhydrous part comprising a composition comprising oxyresveratrol and butylene glycol mixed with a second part comprising water (Composition 11) changed in colour from 1 (at day 1) to 15 (at day 42). This colour change is representative of significant, almost complete, degradation of the oxyresveratrol within the mixed formulation over time.

Sodium metabisulphite has been known to aid aqueous formulation stability, but was found to have little effect on the stability of the formulations of the present invention after mixing. Composition 12a comprising 0.5% oxyresveratrol, butylene glycol, sodium metabisulphite and water were found to change in colour from 1 (at day 1) to 13 (at day 4). The change in colour of the formulation (Composition 12a) is very similar to the change in colour over 6 weeks of Composition 11.

In contrast, it can be seen that a formulation comprising a first anhydrous part comprising a composition comprising oxyresveratrol and butylene glycol mixed with a second part comprising water, sodium metabisulphite and gluconolactone (Composition 12) changed in colour from 1 (at day 1) to 6 (at day 42). This significantly reduced change in colour is indicative of the mixed formulation having significantly improved stability compared to the stability of the mixed formulations of oxyresveratrol, butylene glycol and water, optionally with sodium metabisulphite. The presence of both a sulphur dioxide donor (for example sodium metabisulphite) and a skin conditioning agent (for example gluconolactone) within the mixed formulation appears to significantly reduce and/or minimise the detrimental effect on the stability of the mixed formulation caused by the presence of the water.

### Example 6

### Two Part Formulation Comprising Anhydrous Oxyresveratrol Containing Compositions

A two part formulation was prepared. The first part comprised a composition comprising oxyresveratrol and butylene glycol, and a second part comprises water, sodium metabisulphite, and gluconolactone. The formulation comprises 0.5% oxyresveratrol; 15% butylene glycol; 0.74% gluconolactone; and 0.1% sodium metabisulphite.

It is to be understood that the compositions, formulations, mixed formulations and kits of the present invention may be used in any industry and are not limited to the use for skin lightening. Although the Examples illustrate embodiments in which the formulation comprises two parts it is to be understood that the formulation may comprise any suitable number of parts which may be provided in any suitable number of separate compartments and/or containers. The first and second parts may be mixed and applied simultaneously to a user. The first and second parts may be applied sequentially to a user. One or more of the additional parts may be mixed simultaneously with the first and second parts; mixed with one or more of the first and second parts prior to mixing the first and second parts together; or mixed with the already mixed first and second parts of the formulation. The additional parts may be applied to a user simultaneously with the at least first and second parts of the formulation, or in series prior to or after application of the first and second parts of the formulation. Examples illustrate an embodiment in which the formulation comprises sodium metabisulphite and gluconolatone, it is to be understood that the formulation may comprise any suitable skin conditioning agent and/or sulphur dioxide donor.

1. An anhydrous composition comprising oxyresveratrol and butylene glycol in a cosmetically or pharmaceutically acceptable medium.

2. An anhydrous composition as claimed in claim 1, further comprising one or more additional anhydrous solvents selected from: ethanol, organic solvents, oils, silicones, long-chain alcohols, ketones, ethers, and any combination thereof.

3. An anhydrous composition as claimed in claim 1, in which oxyresveratrol is present within the composition in a concentration in the range of between about 0.00001% and about 20%.

4. An anhydrous composition as claimed in claim 1, in which butylene glycol is present in the composition in a concentration in the range of between about 0.00001% and about 50%.

5. An anhydrous composition as claimed in claim 1, in which the composition consists solely of oxyresveratrol and butylene glycol.

6. A cosmetically or pharmaceutically acceptable formulation comprising at least a first part and a second part, in which the first part comprises an anhydrous composition as claimed in claim 1, and in which the second part comprises one or more solvents selected from anhydrous solvents, aqueous solvents, or a combination thereof.

7. A formulation as claimed in claim 6, in which oxyresveratrol is present within the formulation in a concentration in the range of between about 0.00001% and about 20%.

8. A formulation as claimed in claim 6, in which butylene glycol is present in the formulation in a concentration in the range of between about 0.00001% and about 50%.

9. A formulation as claimed in claim 6, further comprising one or more of: preservatives, skin conditioning agents, stabilisers, antioxidants or sulphur dioxide donors, or any combination thereof.

10. A formulation as claimed in claim 9, further comprising a skin conditioning agent and/or a sulphur dioxide donor.

11. A formulation as claimed in claim 10, in which the skin conditioning agent is gluconolactone and the sulphur dioxide donor is sodium metabisulphite.

12. A formulation as claimed in claim 10, in which the skin conditioning agent and/or sulphur dioxide donor is, or are, each present within the formulation at a concentration of between about 0.0001% and about 30%.

13. A formulation as claimed in claim 9, in which the formulation comprises 0.5% oxyresveratrol; 15% butylene glycol; 0.74% gluconolactone; and 0.1% sodium metabisulphite, and water.

14. An anhydrous composition or a formulation as claimed in claim 1, further comprising one or more of: compositions capable of reflecting or absorbing UV (commonly known as sunscreens), ascorbic acid, a vitamin C derivative, hydroquinone, kojic acid, lactic acid, L-leucine, niacinamide, thiotic acid, a form of arbutin, keratolytic agents such as salicylic acid, physical and chemical exfoliants, superficial, medium and/or deep chemical peels, and any combination thereof.

15. A method of preparing a ready-to-use cosmetically or pharmaceutically acceptable mixed formulation for lightening skin comprising:
obtaining a cosmetically or pharmaceutically acceptable formulation as claimed in claim 6; and
mixing at least the first and second parts of the formulation together to provide a ready-to-use mixed formulation.

16. A kit comprising a formulation as claimed in claim 6, in which the kit comprises a first supply of a first part of the formulation, and at least a second supply of a second part of the formulation, wherein the kit comprises at least two separate chambers, in which the first supply of the first part of the formulation is received within a first chamber, and in which the supply of the second part of the formulation is received within a second chamber.

17. A kit as claimed in claim 16, in which the at least two chambers are provided by a single container or at least two separate containers.

18. A kit as claimed in claim 16, in which at least one of the first and second supplies of the at least two parts of the formulation comprises one or more of a solid impregnated with the first and/or second parts of the formulation respectively.

19. A kit as claimed in claim 18, in which the solid is selected from one or more of a cloth, tissue, or sponge.

20. A kit as claimed in claim 16, in which the kit further comprises one or more of: a sanitising solution, a sterilising solution, an analgesic or anaesthetic composition, a microneedle roller, a replacement microneedle array for the microneedle roller, a moisturising composition, and/or a sunscreen composition, and any combination thereof.

21. Use of a composition or formulation as claimed in claim 1 for skin lightening.
